(19) United States
(12) Patent Application Publication
(10) Pub. No.: US 2023/0079664 A1
(43) Pub. Date: Mar. 16, 2023

(54) FTNIR SPECTROSCOPY FOR REACTION MONITORING OF ACRYLAMIDE SYNTHESIS

(71) Applicant: KEMIRA OYJ, HELSINKI (FI)

(72) Inventors: Petteri SUOMINEN, Helsinki (FI); Marko LAAKKONEN, Helsinki (FI); Samuel OKOLI, Helsinki (FI)

(21) Appl. No.: 17/790,206

(22) PCT Filed: Dec. 23, 2020
(86) PCT No.: PCT/US20/66893

§ 371 Date: Jun. 30, 2022

Related U.S. Application Data
(60) Provisional application No. 62/955,309, filed on Dec. 30, 2019.

(30) Foreign Application Priority Data
Jan. 23, 2020 (FI) 20205067

(51) Int. Cl. C08F 120/56; C07C 231/06; C07C 233/09; C12N 9/14

(52) U.S. Cl. CPC C08F 120/56; C07C 231/06; C07C 233/09; C12N 9/14

(57) ABSTRACT

Provided is a process for producing aqueous acrylamide solution by hydrating acrylonitrile in an aqueous solution in the presence of a biocatalyst, wherein the method comprises in-line monitoring of the acrylamide synthesis reaction by FTNIR spectroscopy. Also provided are an aqueous acrylamide solution obtainable by said process and use thereof for the synthesis of polyacrylamide.

### RELATED APPLICATIONS

The present application claims priority to U.S. Provisional Application No. 62/955,309, filed on Dec. 30, 2019 and Finnish Application No. 20205067 filed on Jan. 23, 2020. The contents of both applications are incorporated by reference in their entireties.

### FIELD OF THE ART

The present disclosure generally relates to the field of acrylamide synthesis, and more particularly, to reaction monitoring of acrylamide synthesis by FTNIR spectroscopy. As such, the present disclosure relates to a process for producing aqueous acrylamide solution by hydrating acrylonitrile in an aqueous solution in the presence of a biocatalyst, wherein the method comprises in-line monitoring of the acrylamide synthesis reaction by FTNIR spectroscopy. The present disclosure also generally relates to aqueous acrylamide solutions obtainable by said process and use thereof for the synthesis of polyacrylamide.

### BACKGROUND

Acrylamide (AMD) has been on the market since the mid-1950s and the acrylamide market has grown steadily since that time. Acrylamide is used primarily in the production of polyacrylamide, which is used in many application fields including water treatment, crude oil recovery, papermaking industry, and mining processes. Acrylamide is produced from acrylonitrile (AN) by hydrolysis reaction in the presence of a catalyst.

Conventionally, production of acrylamide was based on a chemical catalyst process, namely, sulfuric acid-catalyzed hydration reaction or copper-catalyzed hydration which slowly replaced the sulfuric acid process. Enzyme catalyzed acrylamide production processes were developed in the 1980s. Advantages in contrast to the conventional process are low reaction temperature, atmospheric operating pressure, complete conversion, low by-product selectivity and easy downstream processing.

There are two types of nitrile-degradation metabolic pathways existing in microorganisms: nitrilase and nitrile hydratase (NHase) pathways. Three different enzymes are involved in these reactions: nitrilase, (NHase), and amidase.

Nitrilase catalyzes the hydrolysis reaction of nitriles directly into corresponding carboxylic acid and ammonia products. In NHase pathway, NHase and amidase react in series. NHase hydrolyzes nitriles into the corresponding amide product at first. In the presence of amidase, amides may be further converted into corresponding acid and ammonia products. Methods for producing acrylamide from acrylonitrile in the presence of a biocatalyst, e.g. nitrile hydratase are described in numerous patent publications. The monitoring of such reactions, i.e., in order to measure the concentrations of reaction components, including acrylonitrile and acrylamide, as well as by-products (e.g. acrylic acid), in such processes, e.g., using HPLC-based detection methods is also known.

It is an object of the present disclosure to provide an improved process for producing an aqueous acrylamide solution whereby the reaction status is monitored using FTNIR spectroscopy.

### BRIEF SUMMARY

The present disclosure generally relates to an improved process for producing an aqueous acrylamide solution. The process may comprise a) combining water and a biocatalyst having nitrile hydratase activity to provide a slurry; b) feeding acrylonitrile into a reactor comprising said slurry to provide a reaction mixture; and c) monitoring said reaction mixture by in-line FTNIR spectroscopy to measure a concentration of acrylonitrile.

In some embodiments the acrylonitrile feed rate and/or the amount of the water and/or the at least one biocatalyst and/or the temperature may be adjusted during the reaction process based on the detected concentration of acrylonitrile.

In some embodiments, an FTNIR spectrometer probe may be positioned in the reactor.

In some embodiments, an FTNIR spectrometer may be placed outside the reactor. In some specific embodiments, the reactor may comprise a cooling loop connected thereto and an FTNIR spectrometer probe may be positioned in the cooling loop. In some embodiments, an FTNIR spectrometer probe may be positioned in the reactor and another FTNIR spectrometer probe positioned outside the reactor, such as in a cooling loop connected to the reactor. In some embodiments, the FTNIR spectrometer probe placed inside or outside the reactor may be a transflection probe.

In some embodiments, the concentration of acrylonitrile may be within a range of 0 to 10 wt % and may be measured by FTNIR spectroscopy with an accuracy of at least ±1 wt %, more specifically at least ±0.5 wt %, and even more specifically at least ±0.3 wt %.

In some embodiments, the concentration of acrylonitrile may be within a range of 0 to 1 wt % and may be measured by FTNIR spectroscopy with an accuracy of at least ±400 ppm, more specifically at least ±200 ppm, and even more specifically at least ±180 ppm.

In some embodiments, the concentration of acrylonitrile may be within a range of 0 to 1000 ppm and may be measured by FTNIR spectroscopy with an accuracy of at least ±100 ppm, more specifically at least ±80 ppm.

In some embodiments, monitoring said reaction mixture may further comprise measuring a concentration of acrylamide by FTNIR spectroscopy, wherein the concentration of acrylamide may be within a range of 0 to 50 wt % and may be measured with an accuracy of at least ±5 wt %, more specifically at least ±3.8 wt %, and even more specifically at least ±1.3 wt %.

In some embodiments, the final concentration of acrylonitrile as measured by FTNIR spectroscopy may be at most 1000 ppm, at most 500 ppm, at most 250 ppm, or more specifically at most 100 ppm.

In some embodiments, the acrylonitrile feed rate may be adjusted during the process, thereby controlling acrylonitrile accumulation in the reactor.

In some embodiments, 38% to 48% of total amount of acrylonitrile fed to the reactor may be fed during 0 min to 60 min from the beginning of feeding of acrylonitrile into the reactor.

In some embodiments, the reactor may be a semi-batch reactor, a continuous reactor, continuous reactors in series, or stirred tank reactors in series.

In some embodiments, the biocatalyst may comprise 0.1 to 5 kg dry cells/m³ of the reaction mixture.

In some embodiments, the biocatalyst may be a microbe selected from the group consisting of Rhodococcus, Aspergillus, Acidovorax, Agrobacterium, Bacillus, Bradyrhizobium, Burkholderia, Escherichia, Geobacillus, Klebsiella, Mesorhizobium, Moraxella, Pantoea, Pseudomonas, Rhizobium, Rhodopseudomonas, Serratia, Amycolatopsis, Arthrobacter, Brevibacterium, Corynebacterium, Microbacterium, Micrococcus, Nocardia, Pseudonocardia, Trichoderma, Myrothecium, Aureobasidium, Candida, Cryptococcus, Debaryomyces, Geotrichum, Hanseniaspora, Kluyveromyces, Pichia, Rhodotorula, Comomonas, and Pyrococcus or may comprise a combination of at least two of any of the foregoing microbes, more specifically the biocatalyst may be selected from the group consisting of Rhodococcus, Pseudomonas, Escherichia and Geobacillus or may alternatively or in addition comprise a nitrile hydratase derived from any of the foregoing microbes or combinations thereof.

In some embodiments, the biocatalyst may be Rhodococcus rhodochrous or Rhodococcus aetherivorans or a nitrile hydratase derived therefrom.

In some embodiments, said process may further comprise measuring and adjusting temperature of the reaction mixture.

In some embodiments, said process may further comprise maintaining the temperature of the reaction mixture within a range of 15° C. to 25° C.; cooling the reaction mixture when the acrylamide concentration reaches at least 27 wt %, more specifically when it reaches 27 wt % to 38 wt % such that the temperature of the reaction mixture is within a range of 10° C. to 21° C., or within a range of 10° C. to 18° C., when the acrylamide concentration reaches 37 wt % to 55 wt %; and optionally, maintaining the reaction mixture at the temperature range of 10° C. to 21° C., or 10° C. to 18° C., optionally so that the final concentration of acrylonitrile is at most 1000 ppm.

In some embodiments, said process may further comprise cooling the reaction mixture when the acrylamide concentration reaches 28 wt % to 30 wt %.

In some embodiments, said process may further comprise cooling the reaction mixture until the acrylamide concentration reaches 40 wt % to 50 wt %.

In some embodiments, said process may further comprise maintaining the temperature of the reaction mixture at 19° C. to 25° C., more specifically at 20° C. to 22° C., and even more specifically at 22° C.

In some embodiments, said process may further comprise maintaining the temperature of the reaction mixture for 30 min to 120 min.

In some embodiments, said process may further comprise cooling the reaction mixture so that temperature of the reaction mixture is within a range of 10° C. to 16° C., more specifically 13° C. to 16° C., and even more specifically 15° C.

The present disclosure also generally relates to an aqueous acrylamide solution obtainable by a process disclosed herein.

In some embodiments, said aqueous acrylamide solution may be characterized in that:

the concentration of the acrylamide solution may be from 35 wt % to 55 wt %;

concentration of residual acrylonitrile in the acrylamide solution may be equal or less than 1000 ppm, measured by FTNIR; and turbidity of the acrylamide solution may be equal or less than 20 measured from filtrated 0.45 μm acrylamide sample.

In some embodiments, the concentration of residual acrylonitrile in said acrylamide solution measured by FTNIR spectroscopy may be in the range from 0 to 1000 ppm and may be measured with an accuracy of at least ±100 ppm, more specifically at least ±80 ppm.

In some embodiments, the color of said acrylamide solution may be equal or less than 20 measured with spectrophotometer PtCo (455 nm) from 0.45 μm filtrated acrylamide sample.

In some embodiments, the concentration of said acrylamide solution may be from 34 wt % to 55 wt %, more specifically from 38 wt % to 40 wt %.

In some embodiments, the concentration of said acrylamide solution may be from 38 wt % to 55 wt %.

In some embodiments, the concentration of the residual acrylonitrile in said acrylamide solution measured by FTNIR spectroscopy may be equal or less than 100 ppm, more specifically equal or less than 90 ppm, even more specifically equal or less than 50 ppm, still more specifically equal or less than 10 ppm, and yet more specifically 0 ppm.

In some embodiments, the turbidity of said acrylamide solution may be equal or less than 15.

The present disclosure also generally relates to use of said aqueous acrylamide solution obtainable by a process disclosed herein in manufacturing of polyacrylamide.

### DESCRIPTION OF THE DRAWINGS

FIG. 1 presents a schematic of a reactor equipped with an FTNIR spectrometer immersion probe, fiber optic cables, and FTNIR interferometer.

FIG. 2 presents a typical FTNIR absorbance spectrum recorded during the acrylamide synthesis reaction. The inset shows the spectral region where the largest changes occur during the reaction. Absorbance spectra are plotted in dependence of the wave number. The absorbance is defined by A=−log₁₀(I_{T}/I₀). Here, I_{T} and I₀ are the intensities of the transmission and the background spectrum, respectively. In the present case, a spectrum of air was used as background spectrum. This spectrum was recorded when no medium but air was in the optical slit of the fiberoptic probe. The use of the ratio of I_{T} and I₀ has the advantage that the influence of both the transmission path and the characteristic of the measuring system can be compensated.

FIG. 3 presents data related to cross-validation for the AN and AMD concentrations determined by FTNIR and by HPLC for Acrylamide Synthesis Experiment 1 presented in Example 2. Time plotted in the x-axis is shown as time of day (HH:MM).

FIG. 4 presents data related to cross-validation for the AN and AMD concentrations determined by FTNIR and by HPLC for Acrylamide Synthesis Experiment 2 presented in Example 3. Time plotted in the x-axis is shown as time of day (HH:MM).

FIG. 5 presents data related to cross-validation for the AN and AMD concentrations determined by FTNIR and by HPLC for Acrylamide Synthesis Experiment 3 presented in Example 4. Time plotted in the x-axis is shown as time of day (HH:MM).

FIG. 6 presents data related to cross-validation for the AN and AMD concentrations determined by FTNIR (“PLS Experiment) and by HPLC (“Lab”) for Acrylamide Synthesis Experiment 4 presented in Example 5. Time plotted in the x-axis is shown as time of day (HH:MM).

FIG. 7 presents data related to cross-validation for the AN and AMD concentrations determined by FTNIR (“PLS Experiment”) and by HPLC (“Lab”) for Acrylamide Synthesis Experiment 4 presented in Example 5. Time plotted in the x-axis is shown as time of day (HH:MM). The data in these plots are limited to time points in which the concentration of AN was determined to be <1000 ppm.

### DETAILED DESCRIPTION

### I. Overview

According to a first aspect of the present disclosure, there is provided a process for producing an aqueous acrylamide solution. More particularly, there is provided a process for producing an aqueous acrylamide solution comprising combining water and a biocatalyst having nitrile hydratase activity to provide a slurry; feeding acrylonitrile into a reactor comprising said slurry to provide a reaction mixture; and monitoring said reaction mixture by in-line FTNIR spectroscopy to measure a concentration of acrylonitrile. In some embodiments, an FTNIR spectrometer probe may be positioned in the reactor. In some embodiments, the reactor may comprise a cooling loop connected thereto and an FTNIR spectrometer probe positioned in the cooling loop. In some embodiments, the concentration of acrylonitrile may be measured with an accuracy of at least ±100 ppm, more specifically at least ±80 ppm.

According to a second aspect of the present disclosure, there is provided an aqueous acrylamide solution obtainable by said process. More particularly, there is provided an aqueous acrylamide solution characterized in that a concentration of total residual acrylonitrile in the aqueous acrylamide solution is equal to or less than 1000 ppm as measured by FTNIR spectroscopy. The concentration of total residual acrylonitrile may be measured with an accuracy of at least ±100 ppm, more specifically at least ±80 ppm.

In a third aspect of the present disclosure, there is provided use of the aqueous acrylamide solution produced by the disclosed process in manufacturing of polyacrylamide.

It is contemplated that any embodiment discussed in this specification can be implemented with respect to any method, kit, reagent, or composition of the disclosure, and vice versa. Furthermore, compositions of this disclosure can be used to achieve methods of the disclosure.

It will be understood that particular embodiments described herein are shown by way of illustration and not as limitations. The principal features of this disclosure can be employed in various embodiments without departing from the scope of the disclosure. Those skilled in the art will recognize, or be able to ascertain, using no more than routine experimentation, numerous equivalents to the specific procedures described herein. Such equivalents are considered to be within the scope of this disclosure and are covered by the appended claims.

All publications and patent applications mentioned in the instant specification are indicative of the level of skill of one skilled in the art to which this disclosure pertains. All publications and patent applications are herein incorporated by reference to the same extent as if each individual publication or patent application was specifically and individually indicated to be incorporated by reference.

Unless defined otherwise, all technical and scientific terms used herein have the same meaning as is commonly understood by one of skill in the art to which this disclosure belongs. In the event that there are a plurality of definitions for terms herein, those in this section prevail. Where reference is made to a URL or other such identifier or address, it is to be understood that such identifiers can change and particular information on the internet can come and go, but equivalent information can be found by searching the internet. Reference thereto evidences the availability and public dissemination of such information.

As used herein, the singular forms “a,” “an,” and “the” may mean “one” but also include plural referents such as “one or more” and “at least one” unless the context clearly dictates otherwise. All technical and scientific terms used herein have the same meaning as commonly understood to one of ordinary skill in the art to which this disclosure belongs unless clearly indicated otherwise.

As used herein, the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the inherent variation of error for the device, the method being employed to determine the value, or the variation that exists among the study subjects.

As used herein, words of approximation such as, without limitation, “about,” “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 8, 9, 10, 11, 12, 13, 14, or 15%.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, “FTNIR” and “FT-NIR” refer to Fourier Transform Near-Infrared spectroscopy.

As used herein, “biocatalyst” refers to any biocatalyst having nitrile hydratase (NHase) activity. The biocatalyst capable of converting acrylonitrile to acrylamide may be a microorganism which encodes an enzyme having nitrile hydratase activity (e.g, an NHase) or any part of said microorganism having nitrile hydratase activity. With this regard, it is not relevant whether the microorganism is naturally encoding nitrile hydratase, or whether it has been genetically modified to encode said enzyme, or whether a microorganism naturally encoding nitrile hydratase has been modified such as to be able to produce more and/or enhanced nitrile hydratase. Further, it is not relevant whether the enzyme having nitrile hydratase activity is a naturally occurring enzyme or a modified enzyme. The biocatalyst may be selected from said microorganism, lysed cells of said microorganism, a cell lysate of said microorganism, or any combination of these. In a very specific embodiment, the biocatalyst is a nitrile hydratase (NHase).

As used herein “Platinum-Cobalt”, “PtCo” or Pt/Co refers to a color scale that was first introduced in 1892 by chemist Allen Hazen (1869-1930) as a way to evaluate pollution levels in waste water. It has since expanded to a common method of comparison of the intensity of yellow-tinted samples. It is specific to the color yellow and is based on dilutions of a 500 ppm platinum cobalt solution. The color produced by one milligram of platinum cobalt dissolved in one liter of water is fixed as one unit of color in platinum-cobalt scale. The ASTM has detailed description and procedures in ASTM Designation D1209, “Standard Test Method for Color of Clear Liquids (Platinum-Cobalt Scale)”. Color is measured by visual comparison of the sample with platinum-cobalt standards. One unit of color is that produced by 1 mg/L platinum in the form of the chloroplatinate ion. Since very slight amounts of turbidity may interfere with the determination, samples showing visible turbidity are generally clarified by centrifugation. Also, the method is pH dependent.

### II. Reaction Monitoring by FTNIR

The inventors have surprisingly found that the acrylamide synthesis reaction may be monitored by in-line Fourier Transform Near-Infrared Spectroscopy (FTNIR) to achieve an accuracy for the measurement of acrylonitrile concentration in the reaction mixture of at least ±100 ppm (i.e. at least ±80 ppm). This accuracy is an order of magnitude lower than what has been possible by conventional infrared techniques. Accordingly, FTNIR can be used to monitor the maturation phase of the acrylamide synthesis reaction, when the concentration of acrylonitrile is relatively low (e.g. less than 1000 ppm).

The reactor may be any suitable reactor, such as a semi-batch reactor, a continuous reactor, continuous reactors in series, or stirred tank reactors in series, in some exemplary embodiments, a semi-batch reactor. In some embodiments, an FTNIR spectrometer probe may be positioned in the reactor. In some embodiments, the reactor comprises a cooling loop attached thereto and an FTNIR spectrometer probe positioned in the cooling loop. It is contemplated herein that positioning the FTNIR spectrometer probe in the cooling loop would result in reduced air bubbles in the reaction mixture compared to a use of a probe positioned in the reactor. It is further contemplated that this would result in still further improved precision in the reaction component concentration measurements, e.g. in determining the concentration of acrylonitrile.

FTNIR is a non-destructive technique which does not require sample preparation or consumables such as solvents, columns, or reagents. FTNIR also provides real-time analysis, generally requiring 10 seconds per measurement or less, e.g. 1 second per measurement or less than 1 second per measurement. A spectral resolution of 3.2 cm⁻¹ can be achieved when collecting at a rate of 10 seconds per measurement. Accordingly, FTNIR monitoring provides shorter batch cycle time and increased manufacturing capacity compared to conventional wet lab techniques such as HPLC.

FTNIR spectroscopy measures the overtones and combination bands of the molecular vibrations that occur within the NIR range (approximately 780 nm to 2600 nm). As such, FTNIR is more suitable for measuring analytes in aqueous solution compared to FTIR, in which water signals can swamp out those of the analytes. Further, FTNIR spectroscopy is amenable to measuring heterogenous samples whereas FTIR cannot probe beyond the surface of the material so insufficient information is obtained if the material is heterogenous.

The wavelengths used in FTNIR technique enable use of long fiber-optic cables, thus removing the need to put electrical components or ATEX equipment within tens of meters from the reactor.

FTNIR spectrometers are mechanically simpler than traditional dispersive and filter-based NIR instruments because the moving mirror in the FTNIR interferometer is the only continuously moving part in the instrument. Thus, there is very little possibility of mechanical breakdown. In most dispersive instruments, gratings and filters must move in order to generate a spectrum. The benefit of mechanical simplicity is a more reliable, robust scanning mechanism that translates into a more reliable analyzer. Further, conventional NIR techniques are prone to sampling challenges caused by stray light, and have relatively low resolution (16 cm⁻¹ or worse), loss of spectral information, wavelength inaccuracy (which leads to difficulty in transferring methods), and low signal-to-noise ratio. In contrast, FTNIR can provide measurements with a signal-to-noise ratio>10,000.

Dispersive NIR instruments rely on a prism or grating to separate (resolve) the near-infrared frequencies. The best grating can at best separate frequencies 50 cm⁻¹ apart. However, most chemical samples have spectral information that resolves at 8 cm⁻¹. Important spectral information for these types of samples cannot be measured on dispersive instruments so they employ a slit mechanism to achieve higher resolution. Because the slit limits the amount of beam that is measured, a substantial energy loss is incurred, making it difficult or impractical to measure samples at higher resolutions. Because the stroke length of the moving mirror determines resolution on an FTNIR system, there is no degradation of optical throughput as is caused by the slits in dispersive instruments. With no degradation in performance, high resolution spectra can be quickly and easily measured by an FTNIR system. With more spectral information, less reliance is needed on sophisticated chemometric algorithms, which translates into fewer standards being required to develop methods.

The use of an internal reference laser by FTNIR instruments is referred to as Connes Advantage. The benefit of internal calibration is accuracy and precision of better than 0.1 cm⁻¹. Dispersive NIR instruments utilize a mechanically complex prism or grating which cause errors in peak positions and inaccuracies from scan to scan. With the inherent inaccuracy problems of dispersive instruments, reference materials must be employed for calibration. The external calibration has to be repetitively performed, adding difficulty and operator error to measurements. With negligible FTNIR instrumental artifacts due to wavelength inaccuracies, fewer standards are required, providing better results than with dispersive instrumentation.

FTNIR provides the advantage of fiber optic probes. FTNIR probes include classic diffuse reflectance probe for solid materials, transmission immersion probe for clear liquids, and transflection immersion probe for suspensions or emulsions. A transflection immersion probe is exemplified in particular for monitoring aqueous acrylamide synthesis reaction. Various path lengths can be adapted. Various probe materials are available, such as stainless steel, Hastelloy, or ceramics. Moreover, the probe can be customized to different lengths and flange geometries. Accordingly, the FTNIR spectrometer probe may be configured to be positioned in the reactor or in a cooling loop connected to the reactor.

### III. Biocatalyst

The biocatalyst may be fresh (i.e., straight from fermentation); stored, such as stored as frozen (frozen as wet); or dry, before the production of the slurry.

After the fermentation, the biocatalyst slurry may often typically be washed, or otherwise or in addition to suitably treated before entering the slurry or before storage, e.g. by freezing.

The biocatalyst may be any biocatalyst having nitrile hydratase (NHase) activity known in the art.

In accordance with any one of the embodiments of the present disclosure, the biocatalyst capable of converting acrylonitrile to acrylamide may be a microorganism which encodes an enzyme having nitrile hydratase activity (e.g, an NHase) or any part of said microorganism having nitrile hydratase activity. With this regard, it is not relevant whether the microorganism is naturally encoding nitrile hydratase, or whether it has been genetically modified to encode said enzyme, or whether a microorganism naturally encoding nitrile hydratase has been modified such as to be able to produce more and/or enhanced nitrile hydratase. Further, it is not relevant whether the enzyme having nitrile hydratase activity is a naturally occurring enzyme or a modified enzyme. The biocatalyst may be selected from said microorganism, lysed cells of said microorganism, a cell lysate of said microorganism, or any combination of these. In a very specific embodiment, the biocatalyst is a nitrile hydratase (NHase).

Microorganisms encoding nitrile hydratase (e.g. naturally encoding or genetically modified to encode nitrile hydratase) or any part of said microorganism, which can be used as biocatalyst in any one of the embodiments described herein, comprise species belonging to a genus selected from the group consisting of Rhodococcus, Aspergillus, Acidovorax, Agrobacterium, Bacillus, Bradyrhizobium, Burkholderia, Escherichia, Geobacillus, Klebsiella, Mesorhizobium, Moraxella, Pantoea, Pseudomonas, Rhizobium, Rhodopseudomonas, Serratia, Amycolatopsis, Arthrobacter, Brevibacterium, Corynebacterium, Microbacterium, Micrococcus, Nocardia, Pseudonocardia, Trichoderma, Myrothecium, Aureobasidium, Candida, Cryptococcus, Debaryomyces, Geotrichum, Hanseniaspora, Kluyveromyces, Pichia, Rhodotorula, Comomonas, and Pyrococcus. In exemplary embodiments, the biocatalyst is selected from bacteria of the genus Rhodococcus, Pseudomonas, Escherichia, and Geobacillus. Typically, the biocatalyst is selected from the group consisting of Rhodococcus, Aspergillus, Acidovorax, Agrobacterium, Bacillus, Bradyrhizobium, Burkholderia, Escherichia, Geobacillus, Klebsiella, Mesorhizobium, Moraxella, Pantoea, Pseudomonas, Rhizobium, Rhodopseudomonas, Serratia, Amycolatopsis, Arthrobacter, Brevibacterium, Corynebacterium, Microbacterium, Micrococcus, Nocardia, Pseudonocardia, Trichoderma, Myrothecium, Aureobasidium, Candida, Cryptococcus, Debaryomyces, Geotrichum, Hanseniaspora, Kluyveromyces, Pichia, Rhodotorula, Comomonas, and Pyrococcus, or any part of said microorganism having nitrile hydratase activity.

In some embodiments the biocatalyst is selected from the group consisting of Rhodococcus, e.g. Rhodococcus pyridinovorans or Rhodococcus rhodochrous or Rhodococcus aetherivorans, Pseudomonas, Escherichia, and Geobacillus, or any part of said microorganism having nitrile hydratase activity.

In exemplary embodiments, the biocatalyst is Rhodococcus aetherivorans or Rhodococcus rhodochrous, or any part of said microorganism having nitrile hydratase activity.

In one embodiment, the amount of the biocatalyst is 0.1 kg dry cells/m³ to 5 kg dry cells/m³ of reaction mixture.

In another embodiment, the amount of the biocatalyst is from 0.1 g dry cells/kg 100% AMD to 3 g dry cells/kg 100% AMD, based on the final AMD amount, more specifically from 0.2 g dry cells/kg 100% AMD to 3 g dry cells/kg 100% AMD, more specifically 0.2 g dry cells/kg 100% AMD to 2.5 g dry cells/kg 100% AMD. In another embodiment the amount of the biocatalyst is from 0.5 g dry cells/kg 100% AMD to 2 g dry cells/kg 100% AMD or more specifically 1.1 g dry cells/kg 100% AMD to 1.5 g dry cells/kg 100% AMD.

Yet in another embodiment the amount of the biocatalyst is from 0.5 g dry cells/kg 50% AMD to 1 g dry cells/kg 50% AMD, based on the final AMD amount, more specifically from 1.6 g dry cells/kg 50% AMD to 1.8 g dry cells/kg 50% AMD.

Yet in another embodiment the amount of the biocatalyst is 0.1 kg dry cells/m³ to 1.5 kg dry cells/m³ of reaction mixture at the end of the maturation of the reaction mixture. In another embodiment the amount of the biocatalyst is 0.1 kg dry cells/m³ to 1.0 kg dry cells/m³ of reaction mixture.

During the process, more of the biocatalyst may be added, for example, if acrylonitrile starts to accumulate in the reactor. The biocatalyst may be added, for example, as a homogenous slurry in water.

### IV. Reaction Progression

The reaction is conducted at ambient pressure, more specifically at 1 bar.

The slurry may be produced by any known method in the art, such as mixing water and the biocatalyst in a receptacle or in the reactor. More specifically the slurry is homogenous. Strongly agglomerated slurry is less active than homogenous slurry. The biocatalyst is more active in homogenous slurry.

A reaction of acrylonitrile to acrylamide in aqueous solution in the presence of biocatalyst having NHase activity begins once acrylonitrile is fed into a reactor comprising said slurry. Thus, the feeding of acrylonitrile into a reactor comprising said slurry provides a reaction mixture comprising water, acrylamide, acrylonitrile, and biocatalyst.

An aqueous solution of acrylamide in high concentration (e.g. at least 35 wt %, or at least 40 wt %, or at least 45 wt %, or at least 50 wt % or more) can be produced with controlled acrylonitrile feed and process temperature profiles. Cooling of the reactor is typically needed to keep the reaction mixture a desired reaction temperature. The temperature and the acrylonitrile feed rate are each relatively high at the beginning of the reaction to achieve fast reaction rate and short synthesis time. The reactor is started to cool down after, for example, 60 minutes from the start of the reaction since deactivation of the biocatalyst from accumulation of acrylamide is notably lesser in lower temperature compared to higher temperature reaction mixture. The acrylonitrile feed rate is relatively low during the last hours to avoid acrylonitrile accumulation in the reactor.

The feeding of acrylonitrile may be continued throughout the process, more specifically continued throughout the process until the maturation phase. Feed rate of the acrylonitrile may vary during the process. The feeding of acrylonitrile may be continuous or intermittent. The feed rate of acrylonitrile depends on the reaction rate of the acrylonitrile to acrylamide and the rate of biocatalyst deactivation. In one embodiment, feeding of the acrylonitrile is continued throughout the process until the maturation phase.

In one embodiment the acrylonitrile feed rate is adjusted during the process to avoid acrylonitrile accumulation into the reaction mixture. The acrylonitrile is fed during the process with such a rate at which the acrylonitrile converts to acrylamide. More specifically the acrylonitrile amount in the reaction mixture is maintained as less than 3 wt %, or less than 2 wt %, more specifically less than 1 wt %, even more specifically less than 0.5 wt % relative to the total amount of reaction mixture.

In another embodiment of the process, 38% to 48% of total amount of acrylonitrile fed to the reactor is fed during 0 min to 60 min from the beginning of the process; 22% to 30% of total amount of acrylonitrile is fed during 60 min to 120 min of the process; 12% to 18% of total amount of acrylonitrile is fed during 120 min to 180 min of the process; and 8% to 12% of total amount of acrylonitrile is fed during 180 min to 240 min of the process. To reach balance of 100% of fed acrylonitrile, the rest acrylonitrile is fed during the process prior to the maturation phase.

During the maturation phase, substantially no acrylonitrile, and more specifically no acrylonitrile, is fed to the reactor. During maturation, acrylonitrile monomers still present in the reaction mixture react to acrylamide. The reaction mixture is maturated until desired features are reached.

It is known that the biocatalyst starts to deactivate in around 25 wt % to 38 wt % acrylamide solution. See, for example, WO2019/097123. Biocatalyst deactivation caused by acrylamide accumulation is strongly dependent on temperature, and cooling of the reaction mixture notably reduces biocatalyst deactivation. As such, temperature of the reaction mixture is monitored. The monitoring and measuring may be performed with any suitable means and methods in the art.

Initially, the temperature of the reaction mixture is maintained at 15 to 25° C. In one embodiment the temperature is maintained at 19 to 25° C., more specifically at 20 to 22° C. and even more specifically at 22° C. In one embodiment, the temperature is maintained in the desired range by measuring the temperature of the reaction mixture and either cooling the mixture or heating the mixture so that the temperature stays in the desired range. The cooling and/or heating of the reaction mixture may be conducted with known methods in the art.

In some embodiments, the process comprises further cooling of the reaction mixture when the acrylamide concentration reaches at least 27 wt %, more specifically 27 wt % to 38 wt %. In one embodiment the cooling of said reaction mixture is started when the acrylamide concentration reaches 28 wt % to 30 wt %. The cooling of the reaction mixture can be performed by any suitable method and means known in the art, such as by cooling the reactor.

When the cooling of the reaction mixture is started, the temperature of the reaction mixture may be the same, higher, or lower than the temperature of the reaction mixture in the beginning of the process.

In some embodiments, cooling of the reaction mixture is continued so that when the acrylamide concentration reaches 37 wt % to 55 wt %, the temperature of the reaction mixture is within a range of 10° C. to 18° C., or 10° C. to 21° C. In other words, the time period of the cooling of the reaction mixture to the temperature of 10° C. to 18° C., or 10° C. to 21° C., is the time period when the acrylamide concentration of at least 27 wt % (more specifically 27 wt % to 38 wt %) increases to acrylamide concentration 37 wt % to 55 wt % (more specifically 40 wt % to 50 wt %).

In one embodiment, the cooling of the reaction mixture is continued so that so that when the acrylamide concentration reaches 37 wt % to 55 wt %, the temperature is within a range of 10° C. to 16° C., more specifically 13° C. to 16° C., and even more specifically, the temperature is 15° C. In one embodiment the cooling is started, for example, after the reaction mixture has been maintained at 15° C. to 25° C. In one embodiment the reaction mixture is cooled by at least 10° C., more specifically at least 5° C., even more specifically y at least 4° C. The cooling may be conducted linearly or stepwise, typically linearly.

In one embodiment, the reaction mixture is maturated at a temperature within a range of 10° C. to 18° C., or 10° C. to 21° C. when the acrylamide concentration reaches 37 wt % to 55 wt %.

During the maturation, substantially no acrylonitrile, and more specifically no acrylonitrile, is fed to the reactor. During the maturation, unreacted acrylonitrile in the reactor reacts to acrylamide. Maturation begins after the reaction mixture has been cooled and the temperature of the reaction mixture is within the range of 10° C. to 18° C., or 10° C. to 21° C., and/or after the feeding of acrylonitrile into the reactor has ended. More specifically the maturation is continued until final concentration of the acrylonitrile in the reaction mixture is at most 1000 ppm, at most 500 ppm, at most 250 ppm, at most 100 ppm, at most 50 ppm, at most 10 ppm, or at most 0 ppm.

In one embodiment of the process, the temperature of the reaction mixture is maintained at 15° C. to 25° C. for 30 min to 90 min, such as for 45 min to 60 min and the cooling of the reaction mixture to the temperature of 10° C. to 18° C., or 10° C. to 21° C. is performed during a period of time of 45 min to 120 min, such as 60 min to 120 min

As the temperature is kept low at end of the process, less acrylic acid is formed in the process. The activation energy of a reaction forming acrylic acid is higher than the activation energy of the main reaction (formation of acrylamide). The amount of acrylic acid in the aqueous acrylamide solution is at most 300 ppm, more specifically at most 200 ppm, even more specifically at most 100 ppm. Low amount of acrylic acid in the aqueous acrylamide solution is advantageous when cationic polymers are prepared from the acrylamide solution.

The produced aqueous acrylamide solution may be centrifuged to separate acrylamide from biocatalyst.

### IV. Aqueous Acrylamide Solution

In a second aspect of the present disclosure there is provided an aqueous acrylamide solution obtained or obtainable by the process disclosed herein. More particularly there is provided an aqueous acrylamide solution obtained by the process disclosed herein and characterized in that a concentration of total residual acrylonitrile in the aqueous acrylamide solution is equal to or less than 1000 ppm, as measured by FTNIR spectroscopy. In one embodiment the concentration of total residual acrylonitrile in the solution as measured by FTNIR spectroscopy is at most 1000 ppm, at most 500 ppm, at most 250 ppm, at most 100 ppm, at most 90 ppm, more specifically at most 75 ppm, even more specifically at most 50 ppm, and most specifically at most 10 ppm. In one embodiment the concentration of residual acrylonitrile is 0 ppm.

In some embodiments, the concentration of acrylonitrile is measured by FTNIR spectroscopy with an accuracy of at least ±5000 ppm, at least ±3000 ppm, at least ±1000 ppm, at least ±500 ppm, at least ±400 ppm, at least ±300 ppm, at least ±200 ppm, at least ±100 ppm, or at least ±80 ppm.

In some embodiments, the concentration of total residual acrylonitrile is measured by FTNIR spectroscopy with an accuracy of at least ±100 ppm, more specifically at least ±80 ppm.

In some embodiments, the concentration of acrylamide and the concentration of acrylic acid in the aqueous acrylamide solution may also be measured by FTNIR spectroscopy. In some embodiments, the concentration of acrylamide in the aqueous acrylamide solution is from 34 wt % to 55 wt %, or from 38 wt % to 55 wt %, or from 50 wt % to 55 wt %. In some embodiments, the amount of acrylic acid in the aqueous acrylamide solution is at most 300 ppm, more specifically at most 200 ppm, even more specifically at most 100 ppm. Low amount of acrylic acid in the aqueous acrylamide solution is advantageous when cationic polymers are prepared from the acrylamide solution.

In some embodiments, the turbidity of the aqueous acrylamide solution may be equal to or less than 20 as measured by absorbance at 450 nm of a mixture comprising 0.7 ml HCl (0.1 N), 7 ml acetone, and 2.3 ml filtered (0.45 μm) aqueous acrylamide sample. In one embodiment the turbidity of the solution is equal to or less than 15.

In some embodiments, the produced aqueous acrylamide solution may be substantially free of biocatalyst.

### V. Use of Aqueous Acrylamide Solution

In a third aspect of the present disclosure, there is provided use of the aqueous acrylamide solution produced by the disclosed process in manufacturing of polyacrylamide.

### VI. Examples

The following examples are provided for illustrative purposes only and are non-limiting.

### Materials and Methods for Monitoring Acrylamide Synthesis Reaction Used in Examples

Four acrylamide synthesis reactions were performed as described in Examples 1-4. Concentrations of acrylamide (AMD), acrylonitrile (AN), and acrylic acid (AA) were determined by in-line FTNIR spectroscopy and by HPLC for comparative method validation.

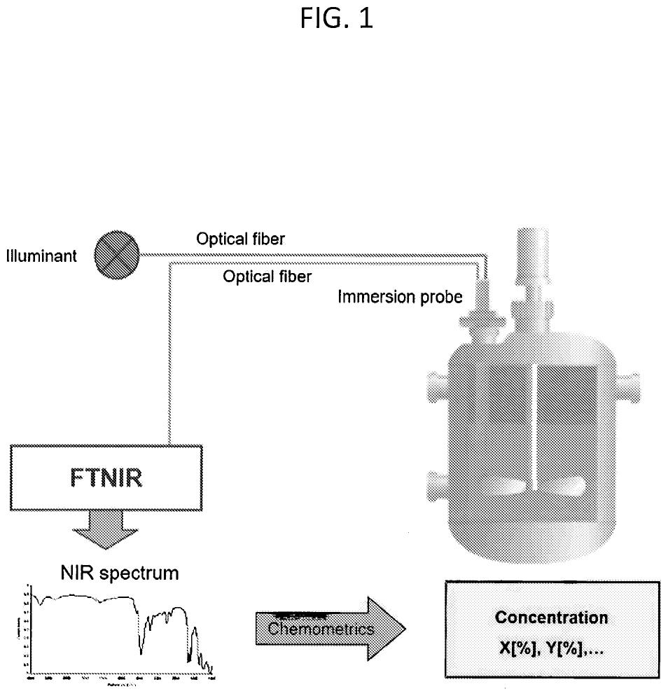

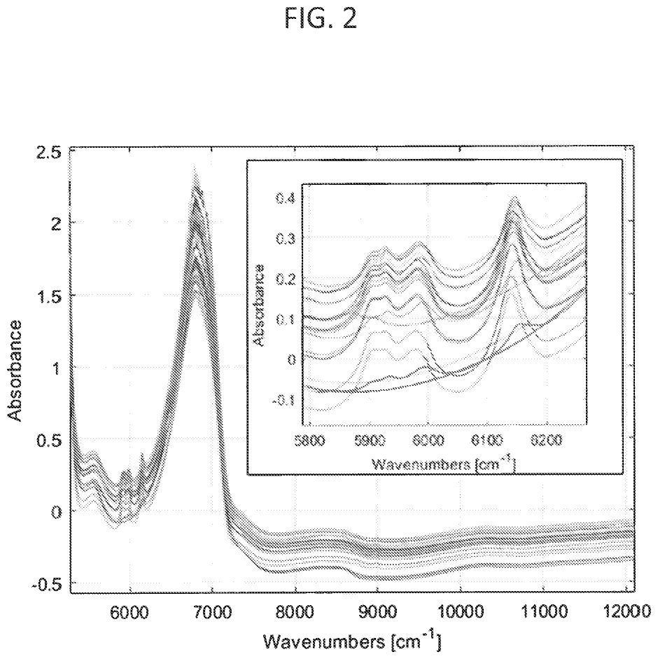

FTNIR spectroscopy was performed using an i-RED FTNIR spectrometer (Infrarot Systeme GmbH, Austria) with a fiber optic-coupled Falcata 12 transflection probe (Hellma, Germany) positioned in a semi-batch reactor. A schematic of the reactor equipped with FTNIR is shown in FIG. 1. The FTNIR Spectrometer parameters are shown in Table 1. A typical FTNIR absorbance spectrum is shown in FIG. 2.

TABLE 1
FTNIR Spectrometer Parameters

| Measurement Principle | Transflection | |
|---|---|---|
| Optical Path Length | 2 | mm |
| Measurement Time | 10 | sec |
| Resolution | 3.2 | cm⁻¹ |
| Wave Number Range | 4260-12000 | cm⁻¹ |

For HPLC measurements, reaction mixture samples (1.5 mL) were removed periodically from the reactor by pipette, filtered through a 0.45 μm PVDF syringe filter and quenched by addition of 10 μL of 0.7 M CuSO₄.5H₂O.

Concentrations of AN, AMD, and AA were determined by HPLC using an 1100 Series HPLC (Agilent Technologies, USA) equipped with a Kintex® 5 μm C18 100 Å LC Column (250×4.6 mm). The components were detected by UV absorbance with a 1260 Diode Array Detector (Agilent Technologies, USA) at 200, 225, and 260 nm depending on each component and its linearity. The flow rate was 1 mL/min in 3.8×10-2 M H3PO4 as eluant. The samples for HPLC were prepared by accurately measuring about 0.2 g of quenched reaction mixture and diluting it into 100 ml of Type 1 MilliQ water which was pretreated with UV radiation for one hour to decompose impurities. A 1961 ppm internal acrylamide standard was run 3 times prior to measurements to ensure that the device was calibrated correctly. Acrylamide 0.1 μL program was used in Agilent software.

### Multivariate Data Analysis

The FTNIR data and the reference values determined by HPLC for the AN and AMD concentrations were subjected to a multivariate data analysis. For AA, no analysis could be performed because all concentrations of this by-product were determined to be 0.0% or <0.01%.

For a quantitative determination of the concentrations of reaction mixture components, the recorded FTNIR spectra together with the reference values from HPLC were subjected to a multivariate data analysis according to the PLS method. Accordingly, the correlation between the reference values for AN and AMD and the FTNIR spectra were examined. In addition, evaluation or prediction models were created in order to calculate the concentrations from the measured FTNIR spectra.

In order to assess the quality (predictive power, stability) of such a model more easily and to better estimate the resulting mean measurement errors, the data were subjected to cross validation. Specifically, in the process of modeling, data samples were omitted in blocks, and the models created with the remaining data/samples are then applied to the omitted data/samples. This ensures that a model is not tested with samples or measurements already included in the model.

In a cross-validation, the two parameters (R² and RMSECV) are given as a measure of the quality of the correlation. These two quantities are explained as follows:
R²: Correlation coefficient. R² is a dimensionless measure of the correlation between the reference values and the FTNIR spectral data. The values are between 0 and 1, with higher values meaning better correlation.
RMSECV: Root mean square error of cross validation. This parameter describes the mean deviation of the FTNIR values from the reference values in the cross validation in the respective units of measure. It indicates the order of magnitude of the expected mean error of a measurement method based on this evaluation model.

Data analysis was performed for each experiment individually and one overall model containing data of all experiments was investigated.

### Example 1: Acrylamide Synthesis Experiment 1

A homogenous slurry was prepared by vortexing 9.96 g thawed biocatalyst in 190 g TRIS buffer (pH 8.0±0.1, TRIS-HCl prepared in DI water) for 15 min at 1000 rpm. The dry cell content of biocatalyst in the slurry was determined to be 2.189 wt %. 570.0 g TRIS buffer (pH 8.0) and 3.09 g biocatalyst slurry were added to a semi-batch reactor equipped with FTNIR spectrometer probe and mixed at 400 rpm to prevent the biocatalyst from settling.

The initial temperature of the reactor was cooled to 21° C. before initializing acrylonitrile feed.

The acrylamide synthesis reaction was started by feeding acrylonitrile to the reactor comprising said slurry to provide a reaction mixture. Over the course of approximately 3 hours, a total of 226.91 g acrylonitrile were fed to the reactor. After 5 hours, the reaction mixture was cooled to 20° C.

Reaction mixture component concentrations determined by HPLC for this experiment are shown in Table 2.

TABLE 2
Concentrations Determined by HPLC with UV Detection

| Time (h:mm) | AN wt % | AMD wt % | AA wt % |
|---|---|---|---|
| 0:00 | 0.00% | 0.00% | 0.00% |
| 0:30 | 1.55% | 10.21% | 0.00% |
| 1:00 | 6.26% | 5.66% | 0.00% |
| 1:30 | 7.36% | 8.38% | 0.00% |
| 2:00 | 8.69% | 11.34% | 0.00% |
| 2:30 | 6.67% | 16.30% | 0.00% |
| 3:07 | 8.71% | 14.15% | 0.00% |
| 3:30 | 10.02% | 16.92% | 0.00% |
| 4:00 | 9.87% | 16.79% | 0.00% |
| 4:32 | 9.74% | 23.91% | 0.00% |

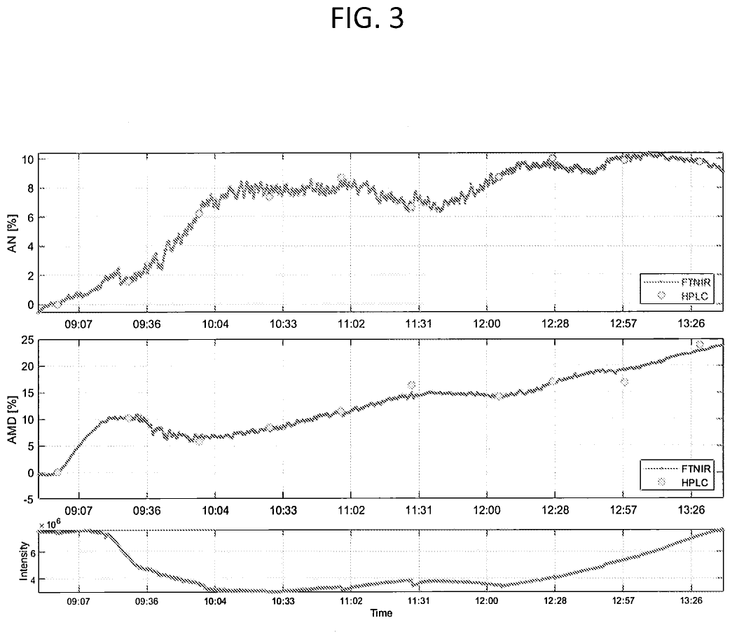

Cross-validation for the AN and AMD concentrations are shown in FIG. 3. The calculated model for the AN concentration shows a good correlation between the reference values and the FTNIR measurement data (R²=0.978). The expected mean measurement error (RMSECV) of a spectroscopic measurement method for the AN content is less than 0.5% in the concentration range from 0 to 10%.

The calculated model for the AMD concentration shows some correlation between the reference values and the FTNIR measurement data (R²=0.715). The expected mean measurement error (RMSECV) of a spectroscopic measurement method for the AMD content is less than 3.5% in the concentration range from 0 to 24%.

### Example 2: Acrylamide Synthesis Experiment 2

In order to determine the accuracy and precision of FTNIR for acrylamide in the concentration range from 0% to 50%, the following reaction protocol was designed to achieve a final acrylamide concentration of at least 50%. In comparison to Acrylamide Synthesis Experiment 1, the amount of acrylonitrile fed to the reaction mixture was increased by 57.3%.

A homogenous slurry was prepared by vortexing 10 g thawed biocatalyst in 190 g DI water at 1000 rpm for 15 min. The dry cell concentration of biocatalyst in the slurry was determined to be 0.669 wt %. DI water (545.6 g) and slurry (17.33 g) were added to a semi-batch reactor equipped with an FTNIR spectrometer probe and mixed at 300 rpm to prevent the biocatalyst from settling.

The initial temperature of the reactor was cooled to 22° C. before initializing acrylonitrile feed.

The acrylamide synthesis reaction was started by feeding acrylonitrile to the reactor comprising said slurry to provide a reaction mixture. A total of 357 g acrylonitrile were fed to the reactor over the course of 3 hours. Specifically, during the first 60 min (i e 0 min to 60 min), 167.8 g (47.0 wt %) acrylonitrile were fed to the reactor. During the next 60 min (i.e. 60 min to 120 min.), 117.8 g (33.0 wt %) acrylonitrile were fed to the reactor. And during the next 60 min (i.e. 120 min to 180 min), 71.4 g (20.0 wt %) were fed to the reactor. The acrylonitrile feed was stopped after 3 h.

After 5 hours, the reaction mixture was cooled to 20° C. The FTNIR probe was then removed and the reaction mixture was purged with air via a porous glass sinter below the liquid surface for 5 min at an air feed rate of 0.5 NL/min. The reaction mixture was then left overnight to continue the AMD maturation phase.

Reaction mixture component concentrations determined by HPLC are shown in Table 3.

TABLE 3
Concentrations Determined by HPLC with UV Detection

| Time (h:mm) | AN wt % | AMD wt % | AA wt % |
|---|---|---|---|
| 0:00 | 0.00% | 0.00% | 0.00% |
| 0:30 | 0.87% | 11.82% | 0.00% |
| 1:00 | 2.23% | 23.99% | 0.00% |
| 2:01 | 0.83% | 33.06% | 0.00% |
| 2:30 | 2.12% | 39.83% | 0.00% |
| 3:00 | 0.73% | 42.72% | 0.00% |
| 3:18 | 1.02% | 46.67% | 0.00% |
| 4:00 | 1.38% | 50.04% | 0.00% |
| 4:40 | 0.05% | 51.34% | 0.00% |
| 5:15 | 0.03% | 54.49% | 0.00% |

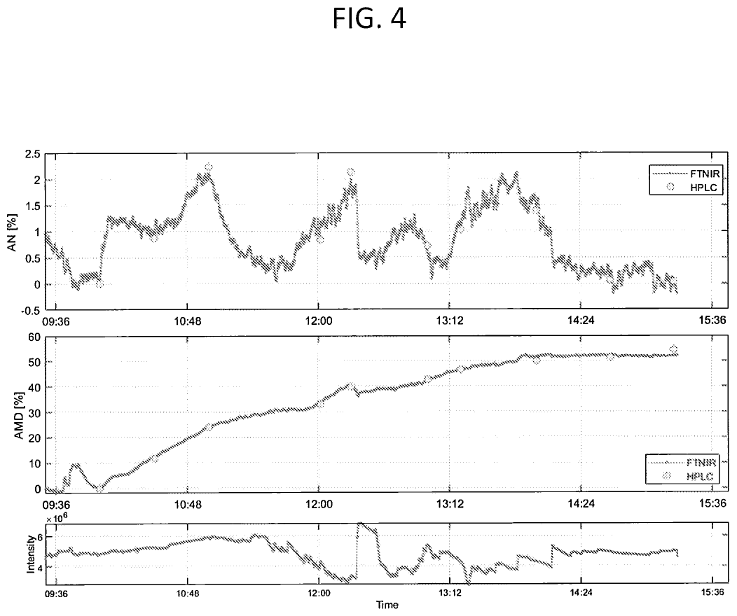

Cross-validation for the AN and AMD concentrations are shown in FIG. 4. The calculated model for the AN concentration shows good correlation between the reference values and the FTNIR measurement data (R²=0.847). The expected mean measurement error (RMSECV) of a spectroscopic measurement method for the AN content from this experiment is less than 0.3%.

The calculated model for the AMD concentration shows very good correlation between the reference values and the FTNIR measurement data (R²=0.966). The expected mean measurement error (RMSECV) of a spectroscopic measurement method for the AMD content from this experiment is less than 3.8%.

### Example 3: Acrylamide Synthesis Experiment 3

In order to determine the accuracy and precision of FTNIR for acrylonitrile in the concentration range from 0% to 1%, the following reaction protocol was used.

A homogenous slurry was prepared by vortexing 10.05 g thawed biocatalyst in 190 g DI water. The dry cell concentration of biocatalyst in the slurry was determined to be 0.819 wt %. DI water (542.5 g) and slurry (20.47 g) were added to a semi-batch reactor equipped with an FTNIR spectrometer probe and mixed at 300 rpm to prevent the biocatalyst from settling.

The initial temperature of the reactor was cooled to 22° C. before initializing acrylonitrile feed.

The acrylamide synthesis reaction was started by feeding acrylonitrile to the reactor comprising said slurry to provide a reaction mixture. During the first 60 min (i.e. 0 min to 60 min), 168 g (47.0 wt %) acrylonitrile were fed to the reactor. During the next 60 min (i.e. 60 min to 120 min), 118 g (33.0 wt %) acrylonitrile were fed to the reactor. During the next 60 min (i.e. 120 min to 180 min), 71 g (20.0 wt %) were fed to the reactor. The acrylonitrile feed was stopped after 3 h. After 5 hours, the reaction mixture was cooled to 20° C.

Reaction mixture component concentrations determined by HPLC are shown in Table 4.

TABLE 4
Concentrations Determined by HPLC with UV Detection

| Time (h:mm) | AN wt % | AMD wt % | AA wt % |
|---|---|---|---|
| 0:00 | 0.00% | 0.00% | 0.00% |
| 0:27 | 0.65% | 13.89% | N.D. |
| 0:57 | 0.42% | 25.24% | N.D. |
| 1:27 | 0.47% | 36.86% | N.D. |
| 2:04 | 0.48% | 44.37% | N.D. |
| 3:04 | 0.53% | 52.94% | N.D. |
| 3:37 | N.D. | 52.03% | N.D. |
| 5:10 | N.D. | 50.65% | N.D. |

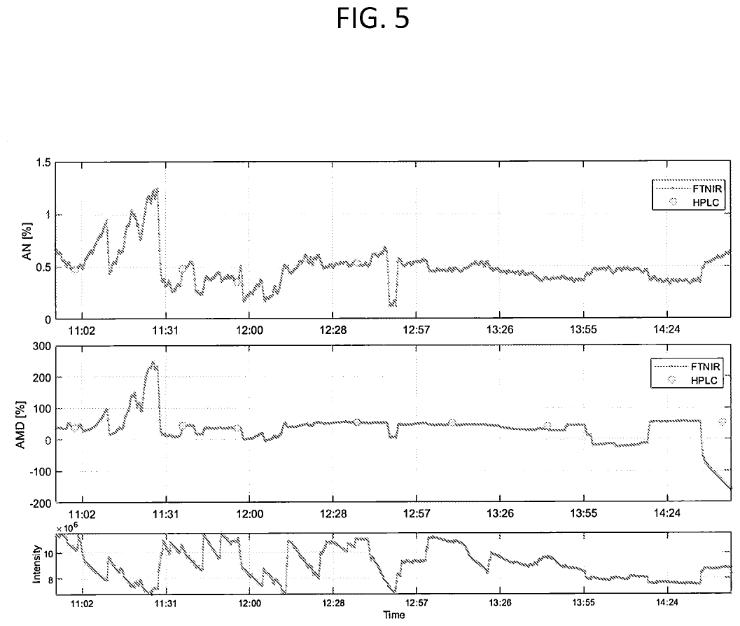

Cross-validation for the AN and AMD concentrations are shown in FIG. 5. The calculated model for the AN concentration shows good correlation between the reference values and the FTNIR measurement data (R²=0.893). The calculated model for the AMD concentration also shows good correlation between the reference values and the FTNIR measurement data (R²=0.898).

The expected mean measurement error (RMSECV) of a spectroscopic measurement from this experiment for the AN content is less than 0.04% (less than 400 ppm) and for the AMD content is less than 1.3%. Although these error values are improved compared to the error levels achieved in Acrylamide Synthesis Experiments 1 and 2, it was noted that air bubbles were present in the reactor. The presence of air bubbles in the reactor are expected to increase the error in the FTNIR measurements relative to FTNIR measurements taken in the absence of air bubbles. As such, it is contemplated that placement of the FTNIR probe outside of the reactor (i.e., in a cooling loop attached to the reactor) will result in further reduced error for FTNIR measurement of the AN and AMD concentrations due to fewer air bubbles in the cooling loop relative to the reactor.

### Example 4: Acrylamide Synthesis Experiment 4

In order to further control the acrylonitrile concentration in the very low concentration range from 0 to 1% (10,000 ppm) and to obtain more data points in this concentration range, the following experiment was designed. Notably, the biocatalyst used in this reaction was denatured and as such, not expected to catalyze acrylamide synthesis. As such, this experiment was a “mock” acrylamide synthesis reaction. In this case, the reactor initially comprised 36.95 wt % acrylamide. A total of only 9.3 g AN were fed to the reactor, causing the AN concentration to gradually increase from 0.0% to 0.99 wt %. As expected, the AMD concentration remained relatively constant. That said, a very slight decrease in the AMD concentration down to 36.58 wt % was observed due to dilution caused by addition of AN.

Reaction mixture component concentrations determined by HPLC are shown in Table 5.

TABLE 5
Concentrations Determined by HPLC with UV Detection

| Time of Day (hh:mm) | AN wt % | AMD wt % | AA wt % |
|---|---|---|---|
| 8:27 | 0.00 | 36.95 | 0.00 |
| 8:30 | 0.00505 | 36.94 | <0.01 |
| 8:33 | 0.010099 | 36.94 | <0.01 |
| 8:36 | 0.015147 | 36.94 | <0.01 |
| 8:39 | 0.020195 | 36.94 | <0.01 |
| 8:42 | 0.025243 | 36.94 | <0.01 |
| 8:45 | 0.03029 | 36.93 | <0.01 |
| 8:48 | 0.040382 | 36.93 | <0.01 |
| 8:51 | 0.050473 | 36.93 | <0.01 |
| 8:54 | 0.060561 | 36.92 | <0.01 |
| 8:57 | 0.070648 | 36.92 | <0.01 |
| 9:00 | 0.080732 | 36.92 | <0.01 |
| 9:22 | 0.090814 | 36.91 | <0.01 |
| 9:25 | 0.100895 | 36.91 | <0.01 |
| 9:28 | 0.110973 | 36.90 | <0.01 |
| 9:31 | 0.121049 | 36.90 | <0.01 |
| 9:34 | 0.131124 | 36.90 | <0.01 |
| 9:37 | 0.141196 | 36.89 | <0.01 |
| 9:40 | 0.151266 | 36.89 | <0.01 |
| 9:43 | 0.181464 | 36.88 | <0.01 |
| 9:46 | 0.211644 | 36.87 | <0.01 |
| 9:49 | 0.241806 | 36.86 | <0.01 |
| 9:52 | 0.27195 | 36.84 | <0.01 |
| 9:55 | 0.302075 | 36.83 | <0.01 |
| 9:58 | 0.332182 | 36.82 | <0.01 |
| 10:01 | 0.362271 | 36.81 | <0.01 |
| 10:04 | 0.392342 | 36.80 | <0.01 |
| 10:07 | 0.422394 | 36.79 | <0.01 |
| 10:10 | 0.452429 | 36.78 | <0.01 |
| 10:13 | 0.482445 | 36.77 | <0.01 |
| 10:16 | 0.512444 | 36.76 | <0.01 |
| 10:19 | 0.542424 | 36.75 | <0.01 |
| 10:22 | 0.572386 | 36.73 | <0.01 |
| 10:25 | 0.60233 | 36.72 | <0.01 |
| 10:28 | 0.632256 | 36.71 | <0.01 |
| 10:31 | 0.662164 | 36.70 | <0.01 |
| 10:34 | 0.692054 | 36.69 | <0.01 |
| 10:37 | 0.721927 | 36.68 | <0.01 |
| 10:40 | 0.751781 | 36.67 | <0.01 |
| 10:43 | 0.781617 | 36.66 | <0.01 |
| 10:46 | 0.811435 | 36.65 | <0.01 |
| 10:49 | 0.841235 | 36.63 | <0.01 |
| 10:52 | 0.871018 | 36.62 | <0.01 |
| 10:55 | 0.900782 | 36.61 | <0.01 |
| 10:58 | 0.930529 | 36.60 | <0.01 |
| 11:01 | 0.960258 | 36.59 | <0.01 |
| 11:04 | 0.989969 | 36.58 | <0.01 |

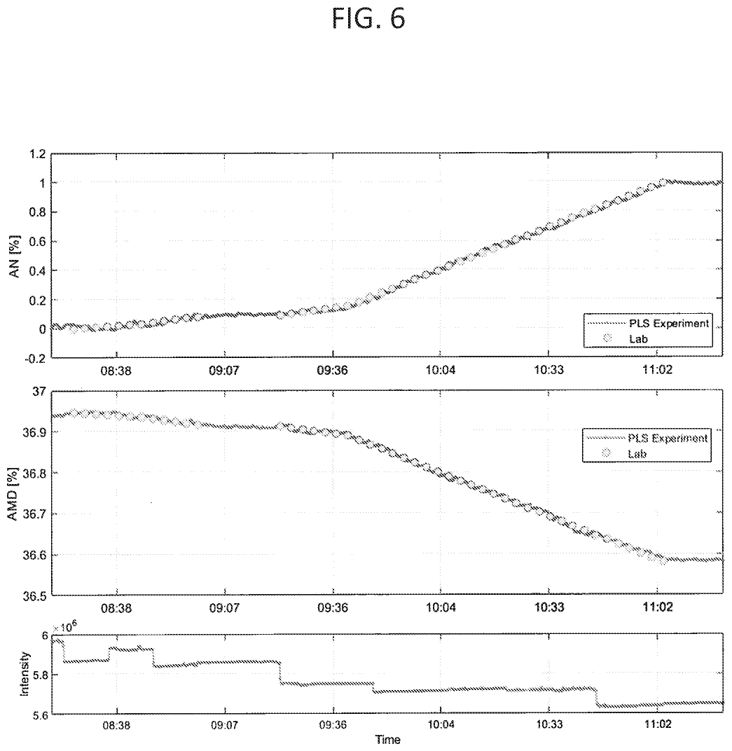

Cross-validation for the AN and AMD concentrations are shown in FIG. 6. The calculated model for the AN concentration shows excellent correlation between the reference values and the FTNIR measurement data (R²>0.99). The expected mean measurement error (RMSECV) of a spectroscopic measurement method for the AN content is less than 0.02% (i.e. less than 0.018% (180 ppm)) in the concentration range from 0 to 1% (0 to 10,000 ppm).

The calculated model for the AMD concentration also shows excellent correlation between the reference values and the FTNIR measurement data (R²>0.99). The expected mean measurement error (RMSECV) of a spectroscopic measurement method for the AMD content is less than 0.01% in the concentration range from 36.6 to 37%.

In order to determine the expected mean measurement error for the acrylonitrile concentration in the very low range from 0 to 0.1% (0-1000 ppm, which is the expected range of acrylonitrile concentration in the produced aqueous acrylamide solution), the data from this experiment were limited to values where [AN]<1000 ppm. A model was developed and tested.

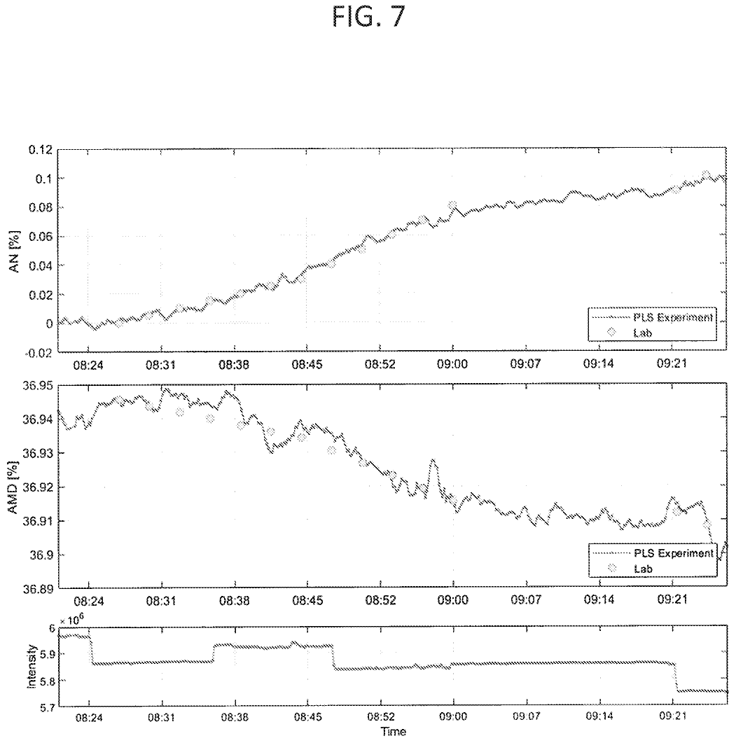

Cross-validation for the AN and AMD concentrations for the [AN]<1000 ppm data are shown in FIG. 7. The calculated model for the AN concentration shows very good correlation between the reference values and the FTNIR measurement data (R²>0.94). The expected mean measurement error (RMSECV) of a spectroscopic measurement method for the AN content is less than 80 ppm in the concentration range from 0 to 1000 ppm.

We claim:

1. A process for producing an aqueous acrylamide solution, comprising:
(a) combining water and at least one biocatalyst having nitrile hydratase activity to provide a slurry;
(b) feeding acrylonitrile into a reactor comprising said slurry to provide a reaction mixture; and
(c) monitoring said reaction mixture by in-line FTNIR spectroscopy to measure a concentration of acrylonitrile.

2. The process of claim 1, wherein one or more of (i) the acrylonitrile feed rate, (ii) the amount of water, (iii) the at least one biocatalyst and/or the amount thereof or (iv) the temperature is adjusted during the reaction process based on the detected concentration of acrylonitrile.

3. The process of claim 1 or 2, wherein an FTNIR spectrometer probe is positioned in the reactor and/or outside the reactor.

4. The process of claim 1, 2 or 3, wherein the reactor comprises a cooling loop connected thereto and an FTNIR spectrometer probe is positioned in the cooling loop.

5. The process of any one of the foregoing claims, wherein the FTNIR spectrometer probe is a transflection probe.

6. The process of any one of the foregoing claims, wherein the concentration of acrylonitrile is within a range of 0 to 10 wt % and is measured by FTNIR spectroscopy with an accuracy of at least ±1 wt %, more specifically at least ±0.5 wt %, and more even more specifically at least ±0.3 wt %.

7. The process of any one of the foregoing claims, wherein the concentration of acrylonitrile is within a range of 0 to 1 wt % and is measured by FTNIR spectroscopy with an accuracy of at least ±400 ppm, more specifically at least ±200 ppm, and more specifically at least ±180 ppm.

8. The process of any one of the foregoing claims, wherein the concentration of acrylonitrile is within a range of 0 to 1000 ppm and is measured by FTNIR spectroscopy with an accuracy of at least ±100 ppm, more specifically at least ±80 ppm.

9. The process of any one of the foregoing claims, wherein monitoring said reaction mixture further comprises measuring a concentration of acrylamide by FTNIR spectroscopy, wherein the concentration of acrylamide is within a range of 0 to 50 wt % and is measured with an accuracy of at least ±5 wt %, more specifically at least ±3.8 wt %, more specifically at least ±1.3 wt %.

10. The process of any one of the foregoing claims, wherein the final concentration of acrylonitrile as measured by FTNIR spectroscopy is at most 1000 ppm, at most 500 ppm, at most 250 ppm, or more specifically at most 100 ppm.

11. The process of any one of the foregoing claims, wherein:
i. the acrylonitrile feed rate is adjusted during the process, thereby controlling acrylonitrile accumulation in the reactor; and
ii. 38% to 48% of the total amount of acrylonitrile fed to the reactor is fed during the time period spanning 0 min to 60 min inclusive from the beginning of feeding acrylonitrile into the reactor.

12. The process of any one of the foregoing claims, wherein the reactor is a semi-batch reactor, a continuous reactor, continuous reactors in series, or stirred tank reactors in series.

13. The process of any one of the foregoing claims, wherein:
i. the biocatalyst comprises 0.1 to 5 kg dry cells/m³ of the reaction mixture;
ii. the biocatalyst is a microbe selected from the group consisting of Rhodococcus, Aspergillus, Acidovorax, Agrobacterium, Bacillus, Bradyrhizobium, Burkholderia, Escherichia, Geobacillus, Klebsiella, Mesorhizobium, Moraxella, Pantoea, Pseudomonas, Rhizobium, Rhodopseudomonas, Serratia, Amycolatopsis, Arthrobacter, Brevibacterium, Corynebacterium, Microbacterium, Micrococcus, Nocardia, Pseudonocardia, Trichoderma, Myrothecium, Aureobasidium, Candida, Cryptococcus, Debaryomyces, Geotrichum, Hanseniaspora, Kluyveromyces, Pichia, Rhodotorula, Comomonas, and Pyrococcus, more specifically the biocatalyst is selected from the group consisting of Rhodococcus, Pseudomonas, Escherichia and Geobacillus or a combination of at least two of any of the foregoing; and/or
iii. the biocatalyst is Rhodococcus rhodochrous or Rhodococcus aetherivorans; and/or
iv. the biocatalyst comprises or additionally comprises a composition comprising a nitrile hydratase derived from any of the foregoing.

14. The process of any one of the foregoing claims, further comprising:
i. measuring and adjusting the temperature of the reaction mixture;
ii. maintaining the temperature of the reaction mixture within a range of 15° C. to 25° C.;
cooling the reaction mixture when the acrylamide concentration reaches at least 27 wt %, more specifically when it reaches 27 wt % to 38 wt % such that the temperature of the reaction mixture is within a range of 10° C. to 21° C., or a range of 10° C. to 18° C., when the acrylamide concentration reaches 37 wt % to 55 wt %; and
optionally, maintaining the reaction mixture at the temperature range of 10° C. to 21° C., or 10° C. to 18° C., further optionally such that the final concentration of acrylonitrile is at most 1000 ppm;
iii. cooling the reaction mixture when the acrylamide concentration reaches 28 wt % to 30 wt %;
iv. cooling the reaction mixture until the acrylamide concentration reaches 40 wt % to 50 wt %;
v. maintaining the temperature of the reaction mixture at 19° C. to 25° C., more specifically at 20° C. to 22° C., and even more specifically at 22° C.;
vi. maintaining the temperature of the reaction mixture for 30 min to 120 min; and/or
vii. cooling the reaction mixture so that temperature of the reaction mixture is within a range of 10° C. to 16° C., more specifically 13° C. to 16° C., and more specifically is 15° C.

15. An aqueous acrylamide solution obtainable by a process of any one of the foregoing claims wherein:
i. it is characterized by the following:
the concentration of the acrylamide solution is from 35 wt % to 55 wt %;
the concentration of residual acrylonitrile in the solution is equal or less than 1000 ppm, measured by FTNIR; and
the turbidity of the solution is equal or less than 20 measured from filtrated 0.45 μm acrylamide sample;
ii. the concentration of residual acrylonitrile in the solution measured by FTNIR spectroscopy is in the range from 0 to 1000 ppm and is measured with an accuracy of at least ±100 ppm, more specifically at least ±80 ppm;
iii. the color of the solution is equal or less than 20 measured with spectrophotometer PtCo (455 nm) from 0.45 μm filtrated acrylamide sample;
iv. the concentration of acrylamide solution is from 34 wt % to 55 wt %, more specifically from 38 wt % to 40 wt %;
v. the concentration of acrylamide is from 38 wt % to 55 wt %;
vi. the concentration of the residual acrylonitrile in the solution measured by FTNIR spectroscopy is equal or less than 100 ppm, more specifically equal or less than 90 ppm, even more specifically equal or less than 50 ppm, still more specifically equal or less than 10 ppm, and most specifically 0 ppm;
vii. the turbidity of the solution is equal or less than 15; or
viii. any combination of the foregoing.

16. Use of the aqueous acrylamide solution of any one of the foregoing claims in the manufacture of polyacrylamide.
